(19) United States
(12) Patent Application Publication
(10) Pub. No.: US 2022/0282264 A1
(43) Pub. Date: Sep. 8, 2022

(54) Endotoxin-free Production of Recombinant Subunit Vaccine Components

(71) Applicant: Ingenza Ltd., Roslin (GB)

(72) Inventors: Ian Fotheringham, Edinburgh (GB); Leonardo Magneschi, Edinburgh (GB); Rita Alexandra Leal Cruz, Edinburgh (GB)

(21) Appl. No.: 17/651,476

(22) Filed: Feb. 17, 2022

Related U.S. Application Data
(60) Provisional application No. 63/150,732, filed on Feb. 18, 2021.

(51) Int. Cl. C12N 15/75; C12N 15/62; A61K 39/12; C12N 15/67; C12P 21/02

(52) U.S. Cl. CPC C12N 15/75; C12N 15/625; A61K 39/12; C12N 15/67; C12P 21/02; C07K 2319/02; C07K 2319/036; C07K 2319/50

(57) ABSTRACT

An endotoxin-free production of recombinant subunit vaccine components, and production methods thereof, using a synthetic virus-like-particle (VLP) to which is attached (and displayed) a fragment of the coronavirus “spike” protein, the Receptor Binding Domain (RBD) and wherein the VLP is produced very effectively using engineered B. subtilis.

### RELATED APPLICATIONS

The present application claims the filing priority of U.S. Application No. 63/150,732 titled “Production Of COVID-19 (SARS-CoV-2) Recombinant Subunit Vaccine Component,” filed on Feb. 18, 2021. The '732 application is hereby incorporated by reference.

### SEQUENCE LISTING

The present specification is being filed with a Sequence Listing in accordance with 37 CFR §§ 1.821 through 1.823. The material of ASCII file titled “Sequence Listing FINAL for Endotoxin-Free Production of Recombinant Subunit Vaccine Component (013001 P0019).txt” of 13,218 bytes, created on May 23, 2022, and submitted via EFS-Web is hereby incorporated by reference.

### TECHNICAL FIELD OF THE INVENTION

The present invention relates to methods for vaccine production. More specifically, the invention relates to production of a recombinant component of a COVID-19 (SARS-CoV-2) protein-subunit vaccine.

### BACKGROUND OF THE INVENTION

As is well-known in the relevant art, viruses are named based on their geneic structure to facilitate the development of diagnostic tests, vaccines, and medicines. Virologists and the wider scientific community do this work, so viruses are named by the International Committee on Taxonomy of Viruses (ICTV). Diseases, on the other hand, are named to enable discussion on disease prevention, spread, transmissibility, severity, and treatment, Human disease preparedness and response is the role of the World Health Organization (WHO), so diseases are officially named by WHO in the. International Classification of Diseases (ICD).

ICTV announced “severe acute respiratory syndrome coronavirus 2 (SARS-CoV2)” as the name of a new virus on Feb. 11, 2020. Concurrently, WHO announced “COVID-19” as the name of this new disease on Feb. 11, 2020, following guidelines previously developed with the World Organization for Animal Health (OIE) and the Food and Agriculture Organization of the United Nations (FAO).

A vaccine for COVID-19 (SARS-CoV-2) was sought thereafter. The vaccine uses a self-assembling synthetic virus-like-particle (VLP) to which is attached (and displayed) a fragment of the coronavirus “spike” protein, the Receptor Binding Domain (RBD). Protein subunit vaccines such as that derived from a VLP with attached RBD domains can offer many advantages over other vaccines that include i) lower cost to produce, ii) more easily scaled-up manufacturing process, iii) re-usability of the delivery vehicle i.e. the VLP versus e.g. alternate viruses, iv) greater stability for storage and/or transportation without need for cold-chain, v) more rapid adaptability to emerging viral variants, vi) simpler use in a multi-valent form to achieve concerted immunity against multiple viruses or viral variants, whereby multiple antigens can be attached and displayed on a single VLP.

A self-assembling VLP refers to a ball-shape protein shell with a diameter of tens of nanometers and well-defined surface geometry that is formed by identical copies of a non-viral protein capable of automatically assembling into a nanoparticle with a similar appearance to a virus particle. Known examples include ferritin (FR), which is conserved across species and forms a 24-mer, as well as viral coat protein (CP3) of the RNA bacteriophage AP205, computationally designed I53-50A and I53-5013, B. stearothermophilus dihydrolipoyl acyltransferase (E2p), Aquifex aeolicus lumazine synthase (LS), and Thermotoga maritima encapsulin, which all form 60-mers. Self-assembling VLP can form spontaneously upon recombinant expression, and optionally secretion to the extracellular medium, of the protein by an appropriate host organism.

VLP can be produced using engineered E. coli, but while E. coli is often favoured due to its rapid manipulability and suitability for scale-up, recombinant proteins manufactured in this microbial host are generally contaminated with endotoxin, a potent immunostimulatory lipopolysaccharide (LPS) molecule, able to induce a pyrogenic response and ultimately trigger septic shock upon introduction to mammals, for example as a contaminating product of a pharmaceutical. Endotoxin contamination can be particularly pronounced when macromolecular structures self-assemble from proteins such as mi3. Separation and removal of bacterial endotoxin from recombinant therapeutic proteins requires complex, challenging, and expensive purification steps that are necessary to ensure the safety of the final product. The Gram-positive bacterium Bacillus subtilis is an alternative prokaryotic host which shares many of the desirable growth, production and scalability characteristics of E. coli as well as holding Generally Recognized as Safe (GRAS) regulatory status, and it does not produce LPS, thereby preventing the risk of this toxin being present in the final product. In addition to its Generally Recognized as Safe (GRAS) status, Bacillus subtilis is widely known for its capacity to produce and secrete large amounts of industrially relevant proteins, and the easy and inexpensive methods for its industrial culture, that can result in very high cell densities.

Some disadvantages associated with recombinant protein expression and secretion in Bacillus subtilis include the reduced structural and/or segregationally stability of certain plasmid vectors in the cell, degradation of protein products by both intracellular and extracellular proteases and the significant variability in extra-cellular secretion levels observed for heterologous proteins when different secretion signal peptides (SP) are fused to the target protein. The optimal signal peptide for one particular recombinant protein is not necessarily the best for the secretion of a different protein. In general, signal peptides and cognate mature proteins have co-evolved to optimize secretion and avoid unfavorable interactions. When a heterologous or non-naturally secreted target is of interest, finding the appropriate signal peptide is a difficult challenge.

Until the disclosed methods of the present application, these and other problems in the prior art may have gone unresolved to some extent by those skilled in the art. The present methods provide results which have shown very high promise of Bacillus subtilis as a production method for VLP as a component of a protein subunit vaccine to protect against COVID-19 which offers much lower cost than competing vaccines and is also much more stable for transportation and/or adaptable to future needs, including large-scale production than competing vaccines.

Bacillus subtilis represents only one of many endotoxin-free organisms that may be suitable for production of VLP, free of contamination with host endotoxin. Other examples include, but are not limited to: other industrially suitable members of the genus Bacillus such as Bacillus licheniformis, Bacillus circulans, Bacillus stearothermophilus; the industrially suitable microbes Corynebacterium glutamicum, Saccharomyces cerevisiae, Pichia pastoris, Aspergillus niger, Aspergillus oryzae, Trichoderma reesei, Streptomyces spp, Lactococcus lactis, Kluyveromyces lactis, Yarrowia lipolytica, Schizosaccharomyces pombe; insect cell lines derived from Spodoptera frugiperda, Spodoptera litura, Estigmene acrea, Danaus plexippus, Trichoplusia ni, Drosophila melanogaster, Bombyx mori; mammalian cell lines such as Chinese Hamster Ovary (CHO) and Human Embryonic Kidney (HEK).

### BRIEF DESCRIPTION OF THE DRAWING

For the purpose of facilitating an understanding of the subject matter sought to be protected, there are illustrated in the accompanying drawing, at least one embodiment thereof, from an inspection of which, when considered in connection with the following description, the subject matter sought to be protected, its construction and operation, and many of its advantages should be readily understood and appreciated.

FIG. 1 is a schematic representation of an embodiment of the disclosed method to produce a VLP-based protein subunit vaccine;

FIG. 2 is a schematic representation of the SpyCatcher-mi3 expression and secretion cassette showing an IPTG-inducible promoter which controls expression of a protein fusion between i) a secretion signal peptide of B. subtilis native secreted protein LytF (Accession number 007532), ii) the SpyCatcher peptide tag, iii) a flexible linker (GSGGSGGS), iv) the mi3 monomer, iv) a short flexible linker (GSG) and v) the affinity purification C-tag (EPEA);

FIG. 3 is a plasmid map containing the expression cassette represented in FIG. 2, the replication initiation protein gene (‘repA’), an ampicillin resistance gene (‘Amp’), the replication origin ‘colE1’ and a chloramphenicol resistance gene (‘Cm’);

FIG. 4 is a chart showing results of densitometry analysis of secreted protein using each of five different secretion signal peptides and a control with no signal peptide;

FIG. 5 is a chart showing the results of densitometry analysis for secretion of mi3 and SpyCatcher-mi3;

FIG. 6 is a chart showing results of densitometry analysis on the impact of codon usage in the level of secreted SpyCatcher-mi3 in B. subtilis;

FIG. 7 is a chart showing post-recovery purity results from SDS-PAGE analysis for levels of contaminants present in SpyCatcher-mi3 by genetic modification of the host;

FIG. 8 is a chart showing results of densitometry analysis on the impact of WprA wall-associated protease in the level of secreted SpyCatcher-mi3 in B. subtilis;

FIG. 9 is a chart showing results of densitometry analysis on the impact of extracellular proteases knockouts on the level and stability of secreted SpyCatcher-mi3;

FIG. 10 is a chart showing serum ELISA results (Area Under the Curve; AUC) demonstrating equivalent immunogenicity of Expi293/E. coli RBD-mi3 (left column of chart) and Pichia/B. subtilis RBD-mi3 (right column of chart);

FIG. 11 is an example of cryo-electron micrograph of RBD-mi3; and

FIG. 12 is a reconstruction of the fully assembled particle representing the VLP scaffold and the RBD antigens.

### DETAILED DESCRIPTION OF A PREFERRED EMBODIMENT

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

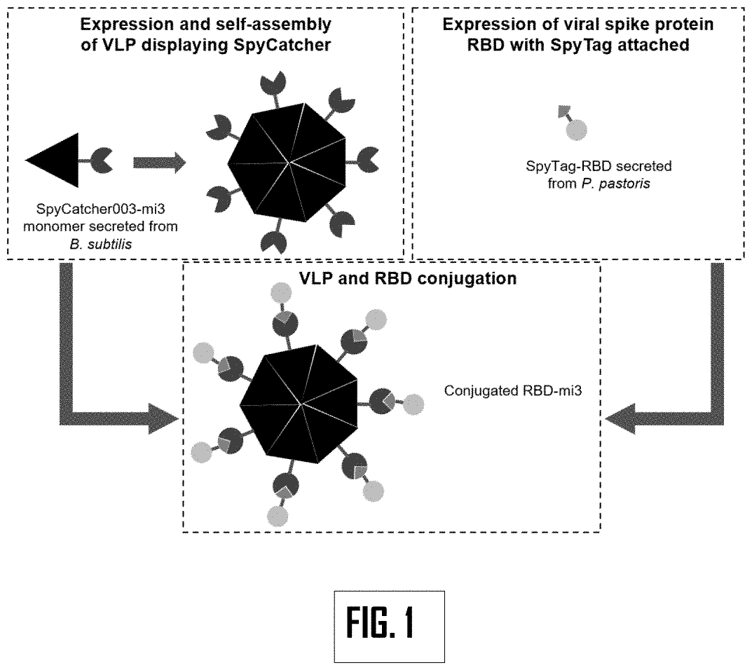

With reference to FIG. 1, an embodiment of the disclosed method is illustrated. The VLP is a self-assembling structure formed by a self-assembling monomer such as “mi3”<SEQ ID NO. 5> which is flanked by a short amino acid linker (peptide) labelled “SpyCatcher”<SEQ ID NO. 3>. The monomer-SpyCatcher peptide is recombinantly expressed and secreted to the extracellular culture medium by B. subtilis and spontaneously assembles into a soccer ball-like dodecahedral structure from which the SpyCatcher tag protrudes in multiple copies. SpyCatcher-mi3 (or homologous sequence) is expressed and secreted using a B. subtilis host before self-assembling in the extracellular environment. By “homologous sequence” it is meant having identity or similarity in primary amino acid sequence to the extent that the protein monomer self-assembles and functions in a comparable way.

SpyTag-RBD is expressed and secreted from a P. pastoris host, as disclosed in co-pending U.S. Patent Application Publication No. US 2021/0206810 A1, titled “Detection of Optimal Recombinants Using Fluorescent Protein Fusions,” filed on Nov. 19, 2020. The '810 Patent Application Publication is hereby incorporated by reference. SpyCatcher-mi3 and SpyTag-RBD conjugate, forming a covalent iso-peptide bond to form a VLP displaying up to 60 copies of the RBD antigen.

The present disclosure focuses on the bacterium Bacillus subtilis, which is known to not produce the endotoxin compound, unlike E. coli. This organism has been chosen because fully functional Virus Like Particle (VLP) production has been clearly exemplified using it.

However, the use of different bacteria, yeast, or other microbes or even mammalian and insect cell systems that do not produce endotoxin may be suitable in place of the Bacillus subtilis. A list of such suitable bacteria, yeast-fungi, insect and mammalian cells may include, but are not limited to, Corynebacterium glutamicum, Saccharomyces cerevisiae, Pichia pastoris, Aspergillus niger, Aspergillus oryzae, Trichoderma reesei, Streptomyces spp, Lactococcus lactis, Kluyveromyces lactis, Yarrowia lipolytica, Schizosaccharomyces pombe, Spodoptera frugiperda, Spodoptera litura, Estigmene acrea, Danaus plexippus, Trichoplusia ni, Drosophila melanogaster, Bombyx mori, Chinese Hamster Ovary (CHO) and Human Embryonic Kidney (HEK) cell lines. Those of skill in the art would recognize the concept of other organisms as the production host, whether or not they may be suitable for other reasons, as within the scope of this disclosure.

Further, as previously noted, there are initially other Bacillus species beyond subtilis that could be valuable and can be tested. A list of such species may include Bacillus licheniformis, Bacillus circulans, and Bacillus stearothermophilus.

Referring to FIG. 1, a property of the SpyCatcher peptide tag is that it can spontaneously and covalently bind to “SpyTag” via an isopeptide bond. Therefore, by using a contiguous peptide that comprises a SpyTag peptide linked to a viral antigen such as the Receptor Binding Domain (RBD) of the SARS-CoV-2 virus, the spontaneous linkage of SpyCatcher and SpyTag can be used to covalently join the VLP to multiple copies of the RBD or other viral antigen by an isopeptide bond. The RBD is made using an engineered Pichia pastoris into which has been introduced DNA to encode the protein sequence that includes the SpyTag-RBD which spontaneously binds to SpyCatcher. This purified VLP and purified RBD bind tightly through the covalent isopeptide bond, forming the vaccine.

In a first example, B. subtilis is used to express and secrete mi3 and SpyCatcher-mi3, a fusion between the peptide tag SpyCatcher and the VLP mi3 monomer protein. B. subtilis is known to be capable of secreting a large variety of proteins, primarily through the major and ubiquitous “Sec” secretion pathway. This organism is very popular for commercial protein production applications, however, despite extensive research, the production of heterologous proteins by B. subtilis is still a hit and miss process, with issues associated with incompatibility between the target protein and the secretion pathway itself. Applicant has addressed the major bottlenecks associated with this pathway to achieve and optimize VLP secretion.

Specifically, it is known that secreted proteins require a secretion signal peptide that targets them to the membrane-bound translocase and that is then removed during the later stages of secretion. In order to produce proteins that are not naturally secreted by the host, such as mi3, a signal sequence needs to be incorporated in-frame with the N-terminus of the target protein. It is generally accepted there is no single optimal Bacillus signal peptide. Applicant has identified a preferable signal peptide capable of successfully targeting mi3 and SpyCatcher-mi3 to the secretion pathway. As well as identifying a suitable signal peptide, codon usage optimization to improve gene expression was demonstrated. It is known gene expression can be improved by accommodating codon bias of the host organism and optimizing mRNA translation initiation and elongation rates. Applicant has also identified a specific gene sequence with a codon usage which allows a significant uplift in secreted VLP from B. subtilis.

This organism, like all Gram-positive bacteria, does not have an outer membrane or a membrane-enclosed periplasm. Although this could be seen as an advantage for heterologous protein production, secreted proteins need to be able to fold rapidly in an environment dominated by the complex physicochemical properties of the peptidoglycan-anionic polymer-protease rich complex that forms the Gram-positive cell wall. Slowly folding proteins can be targeted by several quality control proteases in the membrane and cell wall, as well as extracellular proteases that are present to prevent fatal protein accumulation in these areas of the cell. Additionally, native extracellular proteases, that provide amino acids and peptides as nutrients by degrading proteins in the media, can represent major limitations for the stability of heterologous proteins in the extracellular environment.

In this example, Applicant genetically modified the host organism by knocking out the genes associated with seven feeding proteases in B. subtilis, namely, NprB, AprE, Epr, Bpr, NprE, Mpr and Vpr, and quality control proteases HtrA and HtrB. WprA is a wall-associated protein shown to be involved in degradation of PrsA, a folding chaperone. Applicant has improved both the stability of SpyCatcher-mi3 in the extracellular environment of the cell and its folding efficiency by knocking out eight extracellular protease genes and the wall-associated wprA protease gene to increase PrsA availability, respectively.

The large variety of proteins naturally secreted by B. subtilis can result in a crowded environment from which a heterologous target of interest, such as mi3, needs to be recovered. Initial SpyCatcher-mi3 recovery attempts from the supernatant of B. subtilis cultures showed that a major contaminant, corresponding to the flagellin subunit protein, was consistently present when analyzing the protein content under denaturing conditions. Flagellin, encoded by the hag gene, polymerizes to form the filaments of bacterial flagella, with 12,000 subunits of flagellin making up one flagellum in B. subtilis. Upon deletion of this native gene the major contaminant post SpyCatcher-mi3 purification was eliminated, significantly improving the recovery process.

All the described strategies in this example addressing the bottlenecks in heterologous enzyme secretion allowed the development and optimization of an expression and secretion strain achieving at least 100% increased production over the wild-type B. subtilis host strain.

In another example, Applicant demonstrates how the secreted SpyCatcher-mi3 in B. subtilis can correctly self-assemble into the expected dodecahedron structure comprised of twenty trimers, and conjugate with the SpyTag-RBD fusion expressed and secreted from Pichia pastoris. The iso-peptide bond formation between the “SpyCatcher” and “SpyTag” allows convenient covalent attachment of the antigen at sixty sites on the mi3 particle, resulting in VLP-based protein subunit vaccine particle. Cryo-electron micrographs and subsequent 3D-image reconstruction confirmed the presence of RBD-VLP cages and decoration commensurate with the anticipated appended SpyTag-RBD. Pre-clinical comparisons of immunogenicity in mice injected with RBD-conjugated VLP produced in E. coli and B. subtilis revealed no apparent immunological differences. Applicant thereby developed a production system which is free of E. coli-related endotoxins and simplifies VLP recovery by secretion directly into the extracellular environment.

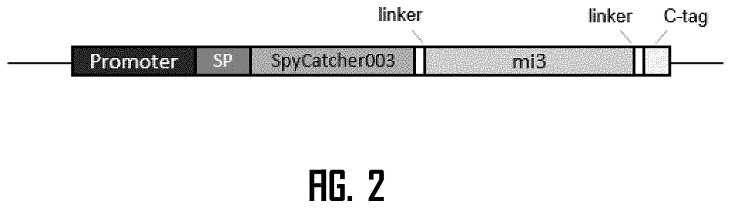

FIG. 2 illustrates a schematic representation of the SpyCatcher-mi3 expression and secretion cassette, which, not including the promoter, is set forth in <SEQ ID NO. 1>. An IPTG-inducible promoter controls the expression of a protein fusion between i) the secretion signal peptide of B. subtilis native secreted protein LytF (Accession number 007532)<SEQ ID NO. 2>, ii) the SpyCatcher peptide tag SEQ ID NO. 3>, iii) a flexible linker (GSGGSGGS)<SEQ ID NO. 4>, iv) the mi3 monomer <SEQ ID NO. 5>, iv) a short flexible linker (GSG)<SEQ ID NO. 6> and v) the affinity purification C-tag (EPEA)<SEQ ID NO. 7>.

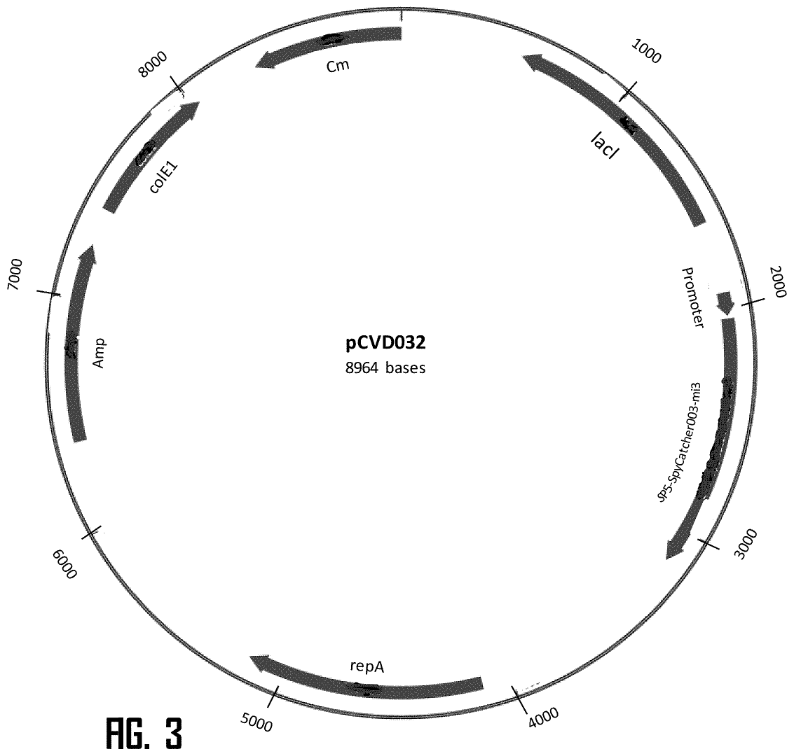

FIG. 3 illustrates a plasmid map containing the expression cassette represented in FIG. 2, the replication initiation protein gene (‘repA’), an ampicillin resistance gene (‘Amp’), the replication origin ‘colE1’ and a chloramphenicol resistance gene (‘Cm’).

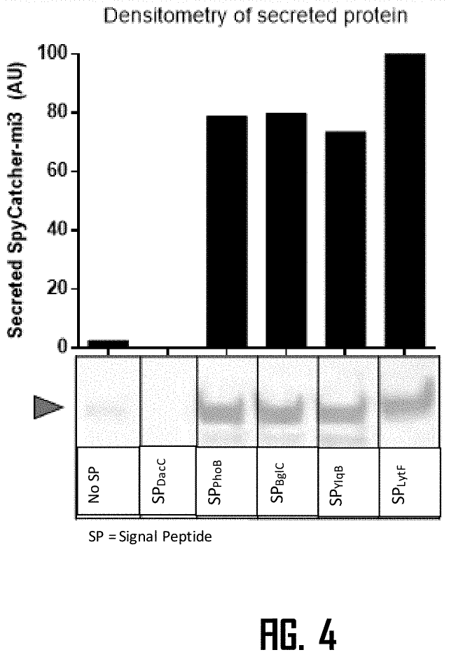

Signal peptide selection: Five different secretion signal peptides were incorporated in-frame with the N-terminus of SpyCatcher-mi3 for targeting to the extracellular environment via the Sec secretion pathway. The tested secretion signal peptides were selected from natively secreted B. subtilis proteins, namely DacC <SEQ ID NO. 8>, PhoB <SEQ ID NO. 9>, BglC <SEQ ID NO. 10>, YlqB <SEQ ID NO. 11> and LytF <SEQ ID NO. 12>. A construct without a signal peptide was also tested as a control. Plasmids carrying the five alternative signal peptide-SpyCatcher-mi3 gene fusions were used to individually transform preparations of competent Bacillus subtilis strain 168. Expression of each alternate gene fusion in transformed Bacillus subtilis 168 was induced for 24 hours in a shake flask culture at 37° C. before harvesting the culture supernatant for recombinant protein yield analysis. Western blot analysis of the secreted SpyCatcher-mi3 from each culture was performed using anti-VLP sera from mice immunized with SpyCatcher-mi3. Densitometry analysis of secreted protein was performed using ImageJ and the results are shown in FIG. 4. It was determined that LytF was the preferred signal peptide.

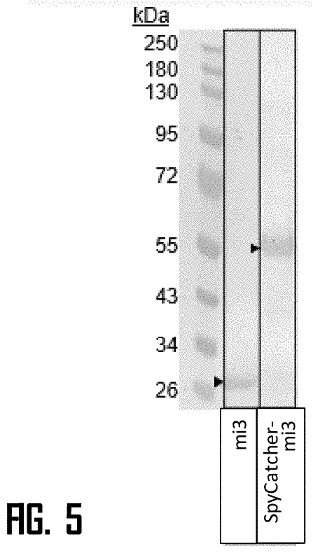

Secretion of mi3 and SpyCatcher-mi3: Plasmids expressing the fusions SP_{LytF}-mi3 and SP_{LytF}-SpyCatcher-mi3 were used to transform Bacillus subtilis 168. Expression was induced for 24 hours in a shake flask at 37° C. before harvesting the culture supernatant for recombinant protein yield analysis. Western blot analysis of secreted mi3 and SpyCatcher-mi3 was performed using anti-VLP sera from mice immunized with SpyCatcher-mi3 and the results are shown in FIG. 5.

Impact of codon usage in the level of secreted SpyCatcher-mi3 in B. subtilis: Plasmids carrying four alternative gene sequences for the protein fusion SP_{LytF}-SpyCatcher-mi3 were used to transform Bacillus subtilis 168. The four alternative gene sequences include Codon usage 1 <SEQ ID NO. 13>, Codon usage 2<SEQ ID NO. 14>, Codon usage 3<SEQ ID NO. 15>, and Codon usage 4<SEQ ID NO. 16>. Expression was induced for 24 hours in a shake flask at 37° C. before harvesting the culture supernatant for recombinant protein yield analysis. Western blot analysis of secreted SpyCatcher-mi3 was performed using anti-VLP sera from mice immunized with SpyCatcher-mi3. Densitometry analysis of secreted protein was performed using ImageJ and the results are shown in FIG. 6. Codon usage No. 3 provided better results than other codon usages tested.

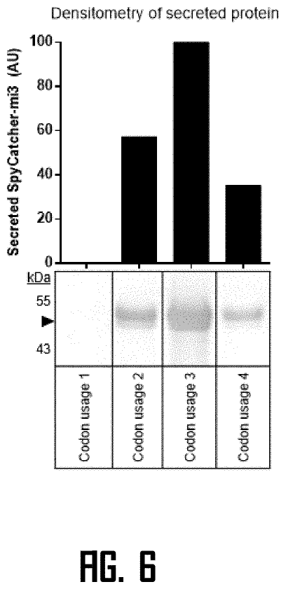

Improvement of SpyCatcher-mi3 post-recovery purity by genetic modification of the host is illustrated in the chart of FIG. 7. A strain containing a knockout mutation of the gene encoding the flagellin subunit protein allows removal of the major contaminant in purified SpyCatcher-mi3 via ammonium sulphate precipitation. Expression was induced for 24 hours in a shake flask at 37° C. before harvesting the culture supernatant and purifying SpyCatcher-mi3 using an ammonium sulphate precipitation method. SDS-PAGE analysis of purified secreted SpyCatcher-mi3 was performed to analyze the level of contaminants present.

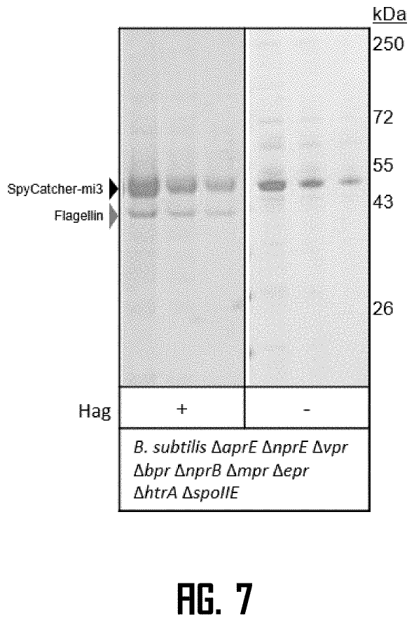

The impact of WprA wall-associated protease in the level of secreted SpyCatcher-mi3 in B. subtilis was also analyzed. The impact of presence or absence of the WprA gene was measured in three different genetic backgrounds, each of which contained knockout mutations in genes encoding either i) none or ii) all 8 of NprB, AprE, Epr, Bpr, NprE, Mpr, Vpr and HtrA or iii) all 8 of NprB, AprE, Epr, Bpr, NprE, Mpr, Vpr and HtrB extracellular proteases. All six strains were transformed with a plasmid identical to FIG. 3 and expression of SpyCatcher-mi3 was induced for 24 hours in a shake flask at 37° C. before harvesting the culture supernatant for recombinant protein yield analysis. Western blot analysis of secreted SpyCatcher-mi3 was performed using anti-VLP sera from mice immunized with SpyCatcher-mi3. Densitometry analysis of secreted protein was performed using ImageJ and the results are shown in FIG. 8.

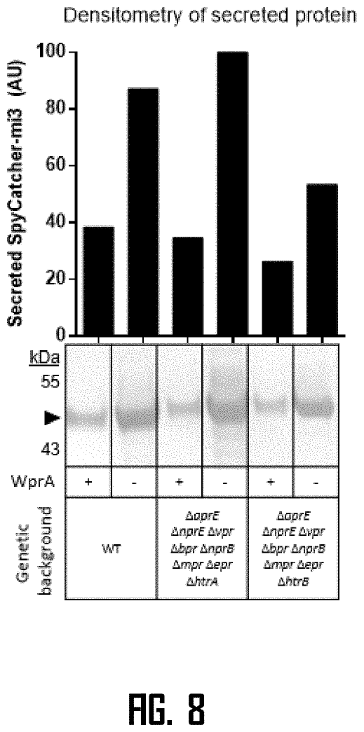

Finally, analysis of the impact of extracellular proteases knockouts on the level and stability of secreted SpyCatcher-mi3 is illustrated in FIG. 9. Three different B. subtilis strains containing knockout mutations in genes encoding i) none or, ii) all 8 of NprB, AprE, Epr, Bpr, NprE, Mpr, Vpr and HtrA or iii) all 9 of NprB, AprE, Epr, Bpr, NprE, Mpr, Vpr, HtrA and WprA extracellular proteases were transformed with a plasmid identical to FIG. 3. Expression was carried out in the Ambr® (Sartorius, Germany) single use fermentor system for up to 39 hours and the level of extracellular SpyCatcher-mi3 was analyzed at different time points post-induction. Western blot analysis of secreted SpyCatcher-mi3 was performed using anti-VLP sera from mice immunized with SpyCatcher-mi3, as shown in FIG. 9.

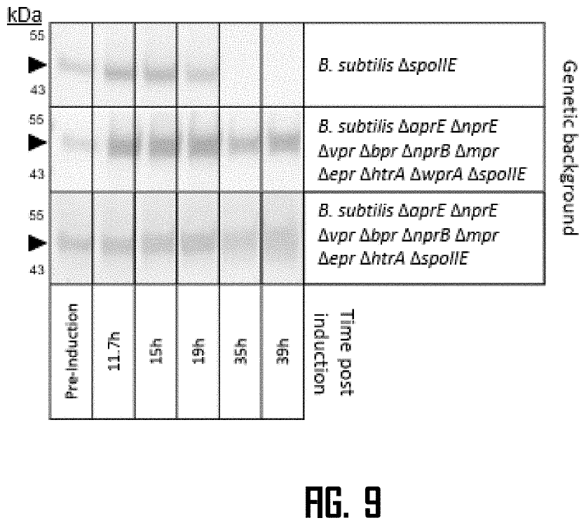

The graph of FIG. 10 shows Serum ELISA results (Area Under the Curve; AUC) demonstrating equivalent immunogenicity of Expi293/E. coli RBD-mi3 and Pichia/B. subtilis RBD-mi3. Female C57Bl/6 mice (n=4) were immunized with homotypic SARS-CoV-2 RBD-mi3 (0.5 μg RBD equivalents) in the indicated producer cells. Data are presented as group means+/−95% confidence intervals. The dotted line represents the lowest serum dilution tested.

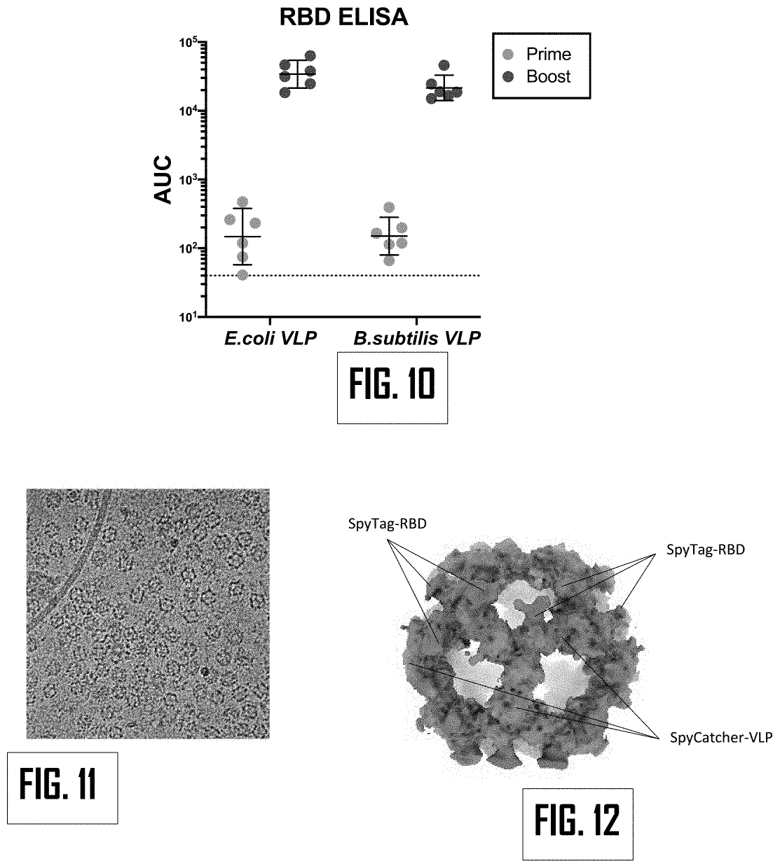

FIG. 11 is an example of cryo-electron micrograph of RBD-mi3 and FIG. 12 is a reconstruction of the fully assembled particle to represent the labeled VLP scaffold and the RBD antigens.

### Materials and Methods

Preparation and transformation of competent Bacillus subtilis. An overnight culture of the strain to be transformed was prepared by inoculating 10 mL LB medium in a 125 mL non-baffled flask, with the appropriate antibiotics where required, and incubated overnight at 37° C., 250 rpm. In a 125 mL flask, 14 mL SM1 medium (Bennallack et al. Journal of Bacteriology, 2014) was inoculated with 1 mL of the overnight culture and grown at 37° C. and 250 rpm until the culture enters stationary phase. 15 mL pre-warmed SM2 medium (Bennallack et al. Journal of Bacteriology, 2014) was added and the culture was grown for a further 90 minutes under the same conditions. In a 15 mL falcon tube, 500 μL of cells were mixed with 200 ng of plasmid DNA and incubated at 37° C., 250 rpm for 30 minutes. 300 μL of LB medium was added and incubated further at 37° C., 250 rpm for 30 minutes. The transformation mixture was spun down at 5,000 xg for 10 minutes and the pellet resuspended in 100 μL of the supernatant before spreading on an LB agar plates with the appropriate antibiotics and incubated overnight at 37° C.

Growth, mi3 and SpyCatcher-mi3 expression in B. subtilis strains in a shake flask. For B. subtilis strains expressing SpyCatcher-mi3, strains were grown from an overnight inoculum in LB medium supplemented with chloramphenicol (10 μg/mL). In the morning, the LB overnight cultures were back diluted to an OD₆₀₀ of 0.05 in 25 mL TB supplemented with 1% (v/v) glycerol and chloramphenicol (10 μg/mL) in a non-baffled 125 mL Erlenmeyer flask. The cultures were then incubated at 37° C., 250 rpm until OD₆₀₀ reached 0.4-0.6. Meanwhile, 100 mL TB (1% v/v glycerol) supplemented with chloramphenicol (10 μg/mL) was prepared, per strain, in a 500 mL non-baffled Erlenmeyer flask and pre-warmed to 37° C. Once OD₆₀₀ 0.4-0.6 was reached, the cultures were back diluted once again, to an OD₆₀₀ of 0.05 in the 100 mL pre-warmed medium and incubated at 37° C., 250 rpm for 2 hours. 1 mL samples were taken for OD₆₀₀ and pre-induction expression levels. Expression was induced with 1 mM IPTG and cultures were incubated at 37° C. and 250 rpm for the indicated amount of time before harvesting.

Growth and SpyCatcher-mi3 expression in B. subtilis strains in the Ambr® 250 bioreactor. For the reactor inoculum 2-20 μL of the appropriate glycerol stock was used to inoculate a 250 ml baffled shake flask containing 50 mL of TB media supplemented with 10 g/L of glycerol and 10 μg/mL of chloramphenicol. The flask was incubated at 37° C. and 250 rpm overnight. After checking the OD₆₀₀ of the flask, the volume of liquid required for a OD₆₀₀ of 0.05 in 150 mL was removed from the flask and centrifuged at 3,900 RPM for 10 minutes in a 50 mL falcon tube. The pellet was then resuspended in the bioreactor batch media and added to the Ambr® 250 vessel to inoculate. The Ambr® 250 microbial vessel had a starting volume of 150 mL of batch media. The pH was controlled at a setpoint of 7 using 2 M H₂SO₄ and 28% NH₄OH (v/v), the dissolved oxygen was maintained at 30% using an agitation cascade of 1,200-4,500 rpm and 1 vvm of air. The temperature setpoint was 37° C. and foam was controlled using polypropylene glycol when required. The feed used was a media feed containing glycerol and 10 g/mL of chloramphenicol. The feeding was started at the point of starvation and used a stepwise feeding profile. The feeding profile was adjusted throughout the fermentations when required. The reactor was induced with 1 mM of IPTG at a OD₆₀₀ of 45-50. The reactor was sampled periodically with sample used for OD₆₀₀ measurement and western blot analysis.

Western blot analysis and relative quantification of mi3 and SpyCatcher-mi3. For western blot analysis of extracellular SpyCatcher-mi3, clarified supernatant samples were first analyzed by SDS-PAGE. Samples were prepared in 1× Bolt™ LDS Sample buffer (Thermo Fisher) with 0.9% (w/v) DTT and denatured at 95° C. for 5 minutes. Standardly, 10 μL of each sample was loaded onto pre-cast Bolt™ BisTris 4-12% polyacrylamide 1 mm thick gels (Thermo Fisher) and electrophoresis was performed in 1×MES buffer (Thermo Fisher) at 200 V for 35 minutes. To indicate molecular weights, 3 μL of Color Prestained Protein Standard, Broad Range (NEB) was included per gel. Western blot analyses were performed following protein transfer from the polyacrylamide gels onto PVDF membranes using the iBlot™ 2 Gel Transfer Device (Thermo Fisher) and iBlot™ 2 PVDF Transfer Stacks, according to manufacturer's instructions. After transfer, membranes were blocked with 5% (w/v) semi-skimmed milk powder in 10 mL phosphate buffered saline (PBS) for 30 minutes. Three 5-minute washes in Tris-Hcl Buffered Saline (TBS) were performed prior to incubating with primary antibody (Anti-Mi3 mouse sera diluted 1:10,000) in TBS for 1 hour at RT, followed by 3×5-minute washes in TBS prior to incubating with secondary antibody (BioRad: Goat Anti-Mouse IgG (H+L)-HRP Conjugate, diluted 1:2000) in TBS for 1 hour at room temperature (RT). Immunodetection was performed, following three 5-minute washes in TBS, using the Thermo Scientific Pierce DAB (3,3′-diaminobenzidine tetrahydrochloride) Substrate Kit. Membranes were rinsed with excess water and dried before scanning. Quantification of SpyCatcher-mi3 was performed by densitometric analysis of the western blot scans using the image processing program, ImageJ.

Ammonium sulphate precipitation of SpyCatcher-mi3 from the supernatant of B. subtilis cultures. Supernatant concentration by Tangential Flow Filtration (TFF): after biomass separation by centrifugation, the supernatant of B. subtilis SpyCatcher-mi3 expressing cultures was first concentrated 10 times by TFF using a 5 kDa hollow fiber TFF filter (Repligen, D06-E005-05-N). A system flow of 216 mL/min (8000 s⁻¹) was used and the trans membrane pressure (TMP) was maintained at approximately 0.8 bar over the course of the concentration. Glycerol was added to the concentrated supernatant to a final concentration of 10% (v/v) before storage at −80° C. until required. Ammonium Sulphate precipitation: concentrated supernatant was defrosted at RT on a tube roller for approximately 45 minutes. Once defrosted, the supernatant was centrifuged at 15,000×g and 4° C. for 45 minutes to pellet any remaining cells. The supernatant was transferred to a fresh beaker and Ammonium Sulphate was added to achieve a final concentration of 10% (w/v). The mixture was then stirred at RT for 1 hour, after which the solution was spun down at 15,000 xg at 4° C. for 45 minutes. The supernatant was decanted, and more Ammonium Sulphate was added in to achieve a final concentration of 20% (w/v). The mixture was stirred again at RT for 1 hour, after which the solution was spun down at 15,000 xg and 4° C. for 45 minutes. The supernatant was discarded, and the pellet was re-suspended in 20 mM Tris:HCl+150 mM NaCl pH 7.6. The solution was concentrated using a 30 kDa spin concentrator (Merck Amicon ultra −15 30K) at 4,500 xg and RT. The retentate from the spin column was mixed with glycerol to achieve a final concentration of 10% (v/v) and the purified SpyCatcher-mi3 solution was stored at −20° C.

Conjugation of RBD-mi3. Affinity purified SpyTag RBD produced in Pichia pastoris was incubated in TBS pH 8.0, overnight and at RT, with SpyCatcher-mi3 produced in either E. coli or B. subtilis. Possible aggregates were then removed by centrifugation at 16,900×g for 30 min at 4° C.

Immunization. Conjugated RBD-mi3 (125 μg/mL) was diluted to 20 μg/mL in TBS and mixed 1:1 (v/v) with AddaVax (InVivogen) prior to immunization. Mice (C57BL/6) were immunized intramuscularly twice with 50 μL of the mixture on day 0 and day 14. Post prime sera was collected on day 13 and post boost sera was collected 3 weeks after the second dose. Anti-RBD ELISA was used to measure the anti-RBD IgG on both post prime and post boost sera.

Cryogenic electro microscopy (Cryo-EM). Conjugated RBD-mi3 was visualized using Cryo-EM and the RBD-mi3 structure was generated from particle picking from the 2D classes followed by 3D classification using three ab-initio models with no symmetry applied.

In addition to the above preferred method for secreting mi3 monomer and the SpyCatcher-mi3 fusion, other monomers may be advantageously secreted and expressed using B. subtilis, and then optimized in the manner described. For example, and not by way of limitation, Helicobacter pylori ferritin (FR), which is conserved across species and forms a 24-mer, as well as viral coat protein (CP3) of the RNA bacteriophage AP205, computationally designed I53-50A and I53-50B, B. stearothermophilus dihydrolipoyl acyltransferase (E2p), Aquifex aeolicus lumazine synthase (LS), and Thermotoga maritima encapsuling.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A method for vaccine or diagnostic applications comprising:
secreting and expressing mi3 monomer from a micro-organism that does not produce endotoxin; and
optimizing the secretion and expression of the mi3 monomer.

2. The method of claim 1, wherein the mi3 monomer is comprised of one of either SpyCatcher-mi3 fusion or a homologous sequence.

3. The method of claim 2, wherein optimizing the secretion of mi3 monomer comprises altering codon usage.

4. The method of claim 3, wherein altering codon usage increases yield of SpyCatcher-mi3 by at least 40%.

5. The method of claim 1, wherein optimizing the secretion of mi3 monomer comprises deletion of a cell wall associated host protease.

6. The method of claim 5, wherein the deletion of a cell wall associated host protease increases yield of SpyCatcher-mi3 by at least 40%.

7. The method of claim 1, further comprising stabilizing the secreted mi3 monomer.

8. The method of claim 2, further comprising stabilizing the secreted mi3 monomer.

9. The method of claim 8, wherein stabilizing the secreted mi3 monomer comprises using extracellular protease knock-outs.

10. The method of claim 1, further comprising improving purification of the secreted mi3 monomer.

11. The method of claim 10, wherein improving purification comprises deleting a host cell gene encoding a major contaminant protein.

12. The method of claim 11, wherein the major contaminant protein comprises flagellin.

13. The method of claim 1, wherein the micro-organism is Bacillus subtilis.

14. The method of claim 2, wherein the micro-organism is Bacillus subtilis.

15. The method of claim 1, wherein the micro-organism is selected from a group consisting of: Bacillus licheniformis, Bacillus circulans, Bacillus stearothermophilus, Bacillus megaterium, Bacillus pumilus, Corynebacterium glutamicum, Saccharomyces cerevisiae, Pichia pastoris, Aspergillus niger, Aspergillus oryzae, Trichoderma reesei, Streptomyces spp, Lactococcus lactis, Kluyveromyces lactis, Yarrowia lipolytica, and Schizosaccharomyces pombe.

16. A method for vaccine or diagnostic applications comprising:
expressing and secreting from a micro-organism that does not produce endotoxin, one of either SpyCatcher-mi3 fusion or a homologous sequence; and
optimizing the secretion and expression of the one of either SpyCatcher-mi3 fusion or homologous sequence.

17. The method of claim 16, wherein optimizing the secretion of one of either SpyCatcher-mi3 fusion or homologous sequence comprises altering codon usage.

18. The method of claim 17, wherein altering codon usage increases yield of the one of either SpyCatcher-mi3 or homologous sequence by at least 40%.

19. The method of claim 16, wherein optimizing the secretion of one of either SpyCatcher-mi3 fusion or homologous sequence comprises deletion of a cell wall associated host protease.

20. The method of claim 19, wherein the deletion of a cell wall associated host protease increases yield of SpyCatcher-mi3 fusion or homologous sequence by at least 40%.

21. The method of claim 16, further comprising stabilizing the secreted one of either SpyCatcher-mi3 fusion or homologous sequence.

22. The method of claim 21, wherein stabilizing the secreted one of either SpyCatcher-mi3 fusion or homologous sequence comprises using a host strain containing knock-out mutations in genes encoding extracellular proteases.

23. The method of claim 16, further comprising improving purification of the secreted one of either SpyCatcher-mi3 fusion or homologous sequence.

24. The method of claim 23, wherein improving purification comprises deleting a gene encoding a major contaminant protein.

25. The method of claim 24, wherein the major contaminant protein comprises flagellin.

26. The method of claim 16, wherein expression and secretion comprises using a signal peptide to direct SpyCatcher-mi3 secretion.

27. The method of claim 26, wherein the signal peptide comprises protein LytF.

28. A method for vaccine or diagnostic applications comprising:
expressing and secreting SpyCatcher-mi3 fusion or homologous sequences from a micro-organism that does not produce endotoxin, the micro-organism being selected from a group consisting of: Bacillus subtilis, Bacillus licheniformis, Bacillus circulans, Bacillus stearothermophilus, Bacillus megaterium, Bacillus pumilus, Corynebacterium glutamicum, Saccharomyces cerevisiae, Pichia pastoris, Aspergillus niger, Aspergillus oryzae, Trichoderma reesei, Streptomyces spp, Lactococcus lactis, Kluyveromyces lactis, Yarrowia lipolytica, and Schizosaccharomyces pombe; and
optimizing the secretion and expression of the one of either SpyCatcher-mi3 fusion or homologous sequences.


SEQUENCE LISTING (16 sequences)

SEQ ID NO: 1 - DNA, 1101 bp, Artificial
  Partial plasmid sequence for virus-like particle (VLP) expression
atgaaaaaaaaactagcagcaggattaacagcaagcgccattgttggcacaacacttgtcgtcacaccggccgaagccggaagctccgtcacaacacttagcggactttccggcgaacaaggcccgtccggcgacatgacaacagaagaggacagcgccacacacatcaagttctccaagcgcgatgaggatggccgcgaacttgctggcgctacaatggaacttcgcgatagctccggcaaaacgatctccacgtggatctccgacggccacgtcaaggacttttatctttatccgggcaaatacacgttcgtcgagacagccgctccagatggctatgaagtcgccacgccgatcgagttcacggtcaacgaggatggacaagtcacagtcgatggcgaagctacagaaggcgatgcccatacgggcggcagcggaggaagcggaggcagcggaggctccatgaagatggaggagcttttcaagaagcacaagatcgtcgccgttcttagagccaactccgtcgaggaagccaagaagaaagctcttgccgttttccttggcggcgtccatcttatcgaaatcacattcacggtcccggatgccgatacggtcatcaaggagctgtcctttcttaaggagatgggcgccattatcggcgccggcacagttacgagcgtcgaacaagctcgcaaagccgtcgaaagcggcgctgagtttatcgtctccccgcaccttgatgaagagatcagccagttcgccaaggagaaaggcgtcttctacatgccgggcgttatgacgccgacggaactggttaaagccatgaagctgggccacacgattctgaaactgtttccgggcgaggtcgtcggaccgcagtttgtcaaagctatgaagggcccgttcccgaatgttaagttcgttccgacgggcggagtcaatcttgacaacgtctgcgagtggttcaaagctggagttcttgccgttggcgttggaagcgcccttgtcaaaggaacgccagtcgaagttgccgagaaggccaaagccttcgtcgagaaaatccgcggctgtacagaaggcagcggcgaaccagaggcctaa

SEQ ID NO: 2 - DNA, 78 bp, Artificial
  DNA sequence within partial plasmid sequence of Seq. ID 1 coding secretion signal peptide native to Bacillius subtillis, modified for expression optimization
atgaaaaaaaaactagcagcaggattaacagcaagcgccattgttggcacaacacttgtcgtcacaccggccgaagcc

SEQ ID NO: 3 - DNA, 360 bp, Artificial
  DNA coding for truncated and engineered FbaB protein from Streptococcus pyogenes, modified for expression optimization
ggaagctccgtcacaacacttagcggactttccggcgaacaaggcccgtccggcgacatgacaacagaagaggacagcgccacacacatcaagttctccaagcgcgatgaggatggccgcgaacttgctggcgctacaatggaacttcgcgatagctccggcaaaacgatctccacgtggatctccgacggccacgtcaaggacttttatctttatccgggcaaatacacgttcgtcgagacagccgctccagatggctatgaagtcgccacgccgatcgagttcacggtcaacgaggatggacaagtcacagtcgatggcgaagctacagaaggcgatgcccatacgggcggcagcgga

SEQ ID NO: 4 - DNA, 24 bp, Artificial
  Designed peptide linker between Seq. ID 3 and Seq. ID 5 in partial plasmid sequence of Seq. ID 1
ggaagcggaggcagcggaggctcc

SEQ ID NO: 5 - DNA, 615 bp, Artificial
  A computationally designed porous dodechahedral structure resulting from a corresponding amino acid sequence as part of partial plasmid sequence in Seq. ID 1
atgaagatggaggagcttttcaagaagcacaagatcgtcgccgttcttagagccaactccgtcgaggaagccaagaagaaagctcttgccgttttccttggcggcgtccatcttatcgaaatcacattcacggtcccggatgccgatacggtcatcaaggagctgtcctttcttaaggagatgggcgccattatcggcgccggcacagttacgagcgtcgaacaagctcgcaaagccgtcgaaagcggcgctgagtttatcgtctccccgcaccttgatgaagagatcagccagttcgccaaggagaaaggcgtcttctacatgccgggcgttatgacgccgacggaactggttaaagccatgaagctgggccacacgattctgaaactgtttccgggcgaggtcgtcggaccgcagtttgtcaaagctatgaagggcccgttcccgaatgttaagttcgttccgacgggcggagtcaatcttgacaacgtctgcgagtggttcaaagctggagttcttgccgttggcgttggaagcgcccttgtcaaaggaacgccagtcgaagttgccgagaaggccaaagccttcgtcgagaaaatccgcggctgtacagaa

SEQ ID NO: 6 - DNA, 9 bp, Artificial
  Designed peptide linker between Seq. ID 5 and Seq. ID 7 in partial plasmid sequence of Seq. ID 1
ggcagcggc

SEQ ID NO: 7 - DNA, 15 bp, Artificial
  Designed artificial peptide tag for affinity chromatography
gaaccagaggcctaa

SEQ ID NO: 8 - DNA, 87 bp, Artificial
  DNA sequence modified for expression optimization of secretion signal peptide (DacC) native to Bacillius subtillis
atgaaaaaaagcatcaagctgtacgtcgccgtccttcttctgtttgttgtcgccagcgtcccgtatatgcatcaagccgcccttgcc

SEQ ID NO: 9 - DNA, 96 bp, Artificial
  DNA sequence modified for expression optimization of secretion signal peptide (PhoB) native to Bacillius subtillis
atgaagaagttcccgaagaaactgctgccgatcgccgttcttagcagcatcgcctttagcagccttgctagcggcagcgttccggaagctagcgcc

SEQ ID NO: 10 - DNA, 87 bp, Artificial
  DNA sequence modified for expression optimization of secretion signal peptide (BglC) native to Bacillius subtillis
atgaagcgcagcatcagcatcttcatcacgtgtcttctgatcacgcttcttacaatgggcggcatgcttgctagcccagctagcgcc

SEQ ID NO: 11 - DNA, 81 bp, Artificial
  DNA sequence modified for expression optimization of secretion signal peptide (YlqB) native to Bacillius subtillis
atgaagaagatcggacttctgttcatgctgtgccttgccgccctttttacaatcggctttccggcccagcaagccgatgcc

SEQ ID NO: 12 - DNA, 78 bp, Artificial
  DNA sequence modified for expression optimization of secretion signal peptide (LytF) native to Bacillius subtillis
atgaaaaaaaaactggccgccggacttacagctagcgccattgttggcacaacacttgtcgtcacaccggccgaagcc

SEQ ID NO: 13 - DNA, 1100 bp, Artificial
  Partial plasmid sequence for virus-like particle (VLP) expression
atgaagaagaagctcgcggcggggctcaccgcgagcgccattgttggcacaacacttgtcgtcacaccggccgaagccggaagctccgtcacaacacttagcggactttccggcgaacaaggcccgtccggcgacatgacaacagaagaggacagcgccacacacatcaagttctccaagcgcgatgaggatggccgcgaacttgctggcgctacaatggaacttcgcgatagctccggcaaaacgatctccacgtggatctccgacggccacgtcaaggacttttatctttatccgggcaaatacacgttcgtcgagacagccgctccagatggctatgaagtcgccacgccgatcgagttcacggtcaacgaggatggacaagtcacagtcgatggcgaagctacagaaggcgatgccatacgggcggcagcggaggaagcggaggcagcggaggctccatgaagatggaggagcttttcaagaagcacaagatcgtcgccgttcttagagccaactccgtcgaggaagccaagaagaaagctcttgccgttttccttggcggcgtccatcttatcgaaatcacattcacggtcccggatgccgatacggtcatcaaggagctgtcctttcttaaggagatgggcgccattatcggcgccggcacagttacgagcgtcgaacaagctcgcaaagccgtcgaaagcggcgctgagtttatcgtctccccgcaccttgatgaagagatcagccagttcgccaaggagaaaggcgtcttctacatgccgggcgttatgacgccgacggaactggttaaagccatgaagctgggccacacgattctgaaactgtttccgggcgaggtcgtcggaccgcagtttgtcaaagctatgaagggcccgttcccgaatgttaagttcgttccgacgggcggagtcaatcttgacaacgtctgcgagtggttcaaagctggagttcttgccgttggcgttggaagcgcccttgtcaaaggaacgccagtcgaagttgccgagaaggccaaagccttcgtcgagaaaatccgcggctgtacagaaggcagcggcgaaccagaggcctaa

SEQ ID NO: 14 - DNA, 1100 bp, Artifical
  Partial plasmid sequence for virus-like particle (VLP) expression
atgaagaaaaaattagccgctgggctcactgcgagcgccattgttggcacaacacttgtcgtcacaccggccgaagccggaagctccgtcacaacacttagcggactttccggcgaacaaggcccgtccggcgacatgacaacagaagaggacagcgccacacacatcaagttctccaagcgcgatgaggatggccgcgaacttgctggcgctacaatggaacttcgcgatagctccggcaaaacgatctccacgtggatctccgacggccacgtcaaggacttttatctttatccgggcaaatacacgttcgtcgagacagccgctccagatggctatgaagtcgccacgccgatcgagttcacggtcaacgaggatggacaagtcacagtcgatggcgaagctacagaaggcgatgccatacgggcggcagcggaggaagcggaggcagcggaggctccatgaagatggaggagcttttcaagaagcacaagatcgtcgccgttcttagagccaactccgtcgaggaagccaagaagaaagctcttgccgttttccttggcggcgtccatcttatcgaaatcacattcacggtcccggatgccgatacggtcatcaaggagctgtcctttcttaaggagatgggcgccattatcggcgccggcacagttacgagcgtcgaacaagctcgcaaagccgtcgaaagcggcgctgagtttatcgtctccccgcaccttgatgaagagatcagccagttcgccaaggagaaaggcgtcttctacatgccgggcgttatgacgccgacggaactggttaaagccatgaagctgggccacacgattctgaaactgtttccgggcgaggtcgtcggaccgcagtttgtcaaagctatgaagggcccgttcccgaatgttaagttcgttccgacgggcggagtcaatcttgacaacgtctgcgagtggttcaaagctggagttcttgccgttggcgttggaagcgcccttgtcaaaggaacgccagtcgaagttgccgagaaggccaaagccttcgtcgagaaaatccgcggctgtacagaaggcagcggcgaaccagaggcctaa

SEQ ID NO: 15 - DNA, 1100 bp, Artificial
  Partial plasmid sequence for virus-like particle (VLP) expression
atgaaaaaaaaactagcagcaggattaacagcaagcgccattgttggcacaacacttgtcgtcacaccggccgaagccggaagctccgtcacaacacttagcggactttccggcgaacaaggcccgtccggcgacatgacaacagaagaggacagcgccacacacatcaagttctccaagcgcgatgaggatggccgcgaacttgctggcgctacaatggaacttcgcgatagctccggcaaaacgatctccacgtggatctccgacggccacgtcaaggacttttatctttatccgggcaaatacacgttcgtcgagacagccgctccagatggctatgaagtcgccacgccgatcgagttcacggtcaacgaggatggacaagtcacagtcgatggcgaagctacagaaggcgatgccatacgggcggcagcggaggaagcggaggcagcggaggctccatgaagatggaggagcttttcaagaagcacaagatcgtcgccgttcttagagccaactccgtcgaggaagccaagaagaaagctcttgccgttttccttggcggcgtccatcttatcgaaatcacattcacggtcccggatgccgatacggtcatcaaggagctgtcctttcttaaggagatgggcgccattatcggcgccggcacagttacgagcgtcgaacaagctcgcaaagccgtcgaaagcggcgctgagtttatcgtctccccgcaccttgatgaagagatcagccagttcgccaaggagaaaggcgtcttctacatgccgggcgttatgacgccgacggaactggttaaagccatgaagctgggccacacgattctgaaactgtttccgggcgaggtcgtcggaccgcagtttgtcaaagctatgaagggcccgttcccgaatgttaagttcgttccgacgggcggagtcaatcttgacaacgtctgcgagtggttcaaagctggagttcttgccgttggcgttggaagcgcccttgtcaaaggaacgccagtcgaagttgccgagaaggccaaagccttcgtcgagaaaatccgcggctgtacagaaggcagcggcgaaccagaggcctaa

SEQ ID NO: 16 - DNA, 1100 bp, Artificial
  Partial plasmid sequence for virus-like particle (VLP) expression
atgaaaaaaaaactggccgccggacttacagctagcgccattgttggcacaacacttgtcgtcacaccggccgaagccggaagctccgtcacaacacttagcggactttccggcgaacaaggcccgtccggcgacatgacaacagaagaggacagcgccacacacatcaagttctccaagcgcgatgaggatggccgcgaacttgctggcgctacaatggaacttcgcgatagctccggcaaaacgatctccacgtggatctccgacggccacgtcaaggacttttatctttatccgggcaaatacacgttcgtcgagacagccgctccagatggctatgaagtcgccacgccgatcgagttcacggtcaacgaggatggacaagtcacagtcgatggcgaagctacagaaggcgatgccatacgggcggcagcggaggaagcggaggcagcggaggctccatgaagatggaggagcttttcaagaagcacaagatcgtcgccgttcttagagccaactccgtcgaggaagccaagaagaaagctcttgccgttttccttggcggcgtccatcttatcgaaatcacattcacggtcccggatgccgatacggtcatcaaggagctgtcctttcttaaggagatgggcgccattatcggcgccggcacagttacgagcgtcgaacaagctcgcaaagccgtcgaaagcggcgctgagtttatcgtctccccgcaccttgatgaagagatcagccagttcgccaaggagaaaggcgtcttctacatgccgggcgttatgacgccgacggaactggttaaagccatgaagctgggccacacgattctgaaactgtttccgggcgaggtcgtcggaccgcagtttgtcaaagctatgaagggcccgttcccgaatgttaagttcgttccgacgggcggagtcaatcttgacaacgtctgcgagtggttcaaagctggagttcttgccgttggcgttggaagcgcccttgtcaaaggaacgccagtcgaagttgccgagaaggccaaagccttcgtcgagaaaatccgcggctgtacagaaggcagcggcgaaccagaggcctaa